(19) United States
(12) Patent Application Publication
(10) Pub. No.: US 2010/0042065 A1
(43) Pub. Date: Feb. 18, 2010

(54) DISPOSABLE ABSORBENT ARTICLE

(76) Inventor: Helene Unschuld, Parkland, FL (US)

Correspondence Address: AMSTER, ROTHSTEIN & EBENSTEIN LLP, 90 PARK AVENUE, NEW YORK, NY, 10016, US

(21) Appl. No.: 12/541,494

(22) Filed: Aug. 14, 2009

Related U.S. Application Data
(60) Provisional application No. 61/088,857, filed on Aug. 14, 2008.

(51) Int. Cl. A61F 13/491

(52) U.S. Cl. 604/385.29; 604/385.01

(57) ABSTRACT

A disposable absorbent undergarment has a front portion, back portion, and a crotch portion. The crotch portion couples the front portion and back portion together. The front portion and back portion are further coupled together, thereby forming a waist opening and leg opening to each side of the crotch portion. Absorbent material is disposed across substantially an entire surface area of the front portion, an entire surface area of the back portion, and an entire surface area of the crotch portion. The front portion, back portion, and crotch portion with absorbent material disposed thereon include contours that substantially conform to an individual's body and include a configuration that reduces bulk under clothing.

### CROSS-REFERENCE TO RELATED APPLICATIONS

This application claims priority to U.S. Provisional Patent Application No. 61/088,857, filed Aug. 14, 2008, the contents of which is incorporated herein by reference.

### FIELD OF THE INVENTION

The present invention generally relates to the field of undergarments, and more particularly relates to women's undergarments.

### BACKGROUND OF THE INVENTION

Conventional women's undergarments generally do not provide sufficient fluid/moisture barriers during a woman's menstrual cycle. Therefore, panty liners or other feminine care products are generally required to be used with conventional undergarments. One problem with this combination is that the feminine care product is sometimes unable to provide a full protection barrier from the fluid/moisture which can lead to leaks and staining of the undergarment and clothing of the individual.

To overcome some of these problems, undergarments have been created that include absorbent regions in areas where feminine care products are placed or that are in close proximity to feminine products on/in a woman's body. These undergarments help prevent any leakage. However, because these undergarments are designed to be used with feminine products they generally do not include strong absorbent properties that would prevent leakage and staining if a feminine product is not used in combination therewith. If a woman is caught off-guard these under garments provide minimal protection from leakage and staining.

Furthermore, current undergarments that include absorbent materials generally only include absorbent material in limited areas of the undergarment. The lack of additional absorbent areas can lead to various problems. For example, when fluid absorbs into these limited areas and reaches the end of the absorbent material, leaks and staining can still occur.

### SUMMARY OF THE INVENTION

In one embodiment, a disposable absorbent undergarment is disclosed. The absorbent undergarment has a front portion, back portion, and a crotch portion. The crotch portion couples the front portion and back portion together. The front portion and back portion are further coupled together, thereby forming a waist opening and leg opening to each side of the crotch portion. Absorbent material is disposed across substantially an entire surface area of the front portion, an entire surface area of the back portion, and an entire surface area of the crotch portion. The front portion, back portion, and crotch portion with absorbent material disposed thereon include contours that substantially conform to an individual's body and include a configuration that reduces bulk under clothing.

### BRIEF DESCRIPTION OF THE DRAWINGS

The accompanying figures where like reference numerals refer to identical or functionally similar elements throughout the separate views, and which together with the detailed description below are incorporated in and form part of the specification, serve to further illustrate various embodiments and to explain various principles and advantages all in accordance with the present invention, in which:

FIG. 1 is a top view of a preassembled panty configuration according to one embodiment of the present invention;

FIG. 2 is a side perspective view of an assembled panty configuration according to one embodiment of the present invention; and

FIG. 3 is a front perspective view of an assembled panty configuration being worn by an individual according to one embodiment of the present invention.

### DETAILED DESCRIPTION OF THE EXEMPLARY EMBODIMENTS

As required, detailed embodiments of the present invention are disclosed herein; however, it is to be understood that the disclosed embodiments are merely examples of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure and function. Further, the terms and phrases used herein are not intended to be limiting; but rather, to provide an understandable description of the invention.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

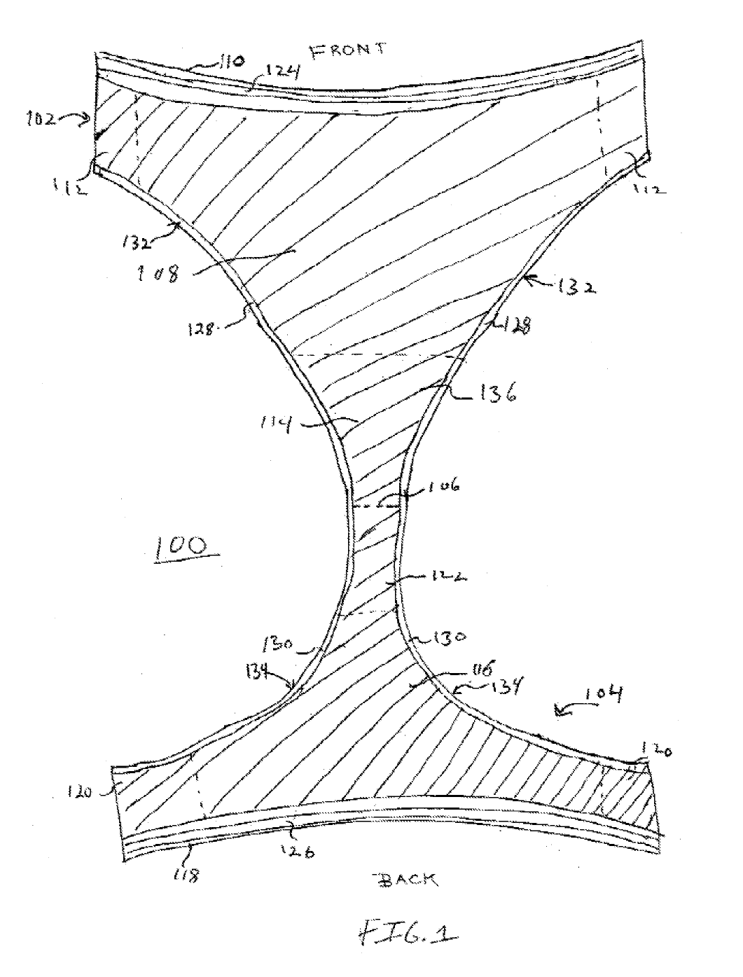

FIG. 1 shows a disposable absorbent undergarment 100 in a flat configuration prior to assembly. In particular, FIG. 1 shows the inner portions of the undergarment 100. However, it should be noted that the following discussion with respect to FIG. 1 is also applicable to the outer portions (the portions visible when an individual is wearing the undergarment 100) of the undergarment 100 as well.

The undergarment 100, in one embodiment, is a woman's undergarment that can be of any style such as bikini, string bikini, low-rise string bikini, hipster, thong, briefs, boy-shorts, or any other style of undergarment. The undergarment 100 includes a front portion 102 and a back portion 104. The front and back portions 102, 104 are generally situated on the front and back portions, respectively of the individual wearing the undergarment 100. Although FIG. 1 shows undergarment 100 fabricated from a continuous piece of material the undergarment can be fabricated from multiple pieces as well. For example, the front portion 102 and the back portion 104 can be separate pieces and coupled together at one or more points such as an area indicated by the dashed line 106.

The front portion 102 includes a front body region 108, a front waist region 110, a hip region 112, and a feminine region section 114. The back portion 104 includes a back body region 116, a back waist region 118, a hip region 120, and a feminine region section 122. The waist regions 110, 118 each include stretchable material 124, 126 such as (but not limited to) elastic, spandex, or the like. The stretchable material 124, 126 secures the undergarment 100 to the individual's body. Each of the front portion 102 and the back portion 104 also include stretchable material 128, 130 such as (but not limited to) elastic, spandex, or the like disposed along an outer region or edge 132, 134 of the undergarment 100. This outer region stretchable material 128, 130 secures the undergarment along the legs and buttocks of an individual.

The undergarment 100, in one embodiment, comprises one or more absorbent materials 136 disposed throughout. For example, each of the front body region 108, front waist region 110, hip region 112, and feminine region section 114 of the front/back portions 102, 104 (of both the inner and outer areas of the undergarment 100) are comprised of absorbent material(s). In other words, the entire undergarment 100 is made from absorbent material as denoted by the diagonal lines 136. It should be noted that the absorbent material 136 can also be disposed over the stretchable material 128, 130 and/or connected to the stretchable material 128, 130.

The absorbent material 136 retains any leakage that a woman may experience and acts a protective barrier so that seepage onto other clothing articles does not occur. An advantage of having substantially the entire undergarment 100 fabricated from the absorbent material 136 is that a larger surface area is provided for absorption/protection than in conventional ladies protective undergarments.

Another advantage is that the undergarment 100 is disposable and does not require additional feminine products to be used in combination therewith. For example, a woman is not required to place any type of liner within the feminine region 114, 122 to provide a protective barrier from fluids. Also, because substantially the entire undergarment 100 is comprised of absorbent material(s) a woman can simply dispose of the undergarment 100 when needed.

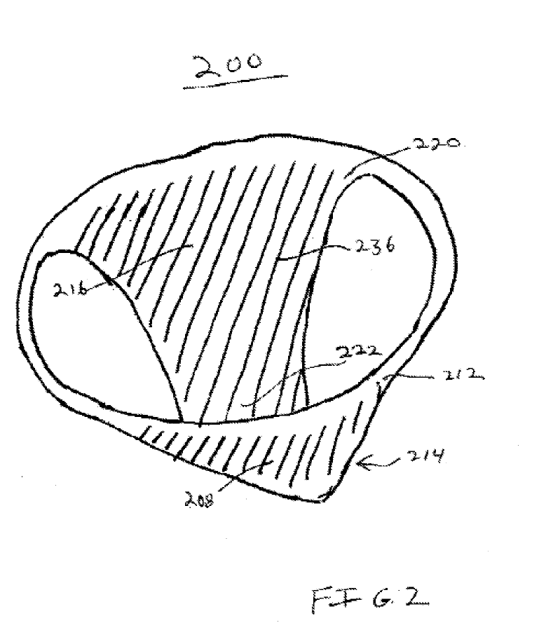

The undergarment 100, in one embodiment, is configured to resemble a conventional non-absorbent undergarment that women wear almost every day. For example, the undergarment 100 has the same cut, lines, and general thickness as a conventional pair of non-absorbent panties. In other words, the absorbent material 106 does not add uncomfortable bulkiness such as that found with disposable diapers. For example, FIG. 2 shows one embodiment of an assembled pair of an absorbent disposable undergarment 200 similar to the undergarment discussed above with respect to FIG. 1. As can be seen, the undergarment 200 of FIG. 2 resembles a conventional pair of ladies non-disposable panties. However, the body regions 208, 216, hip regions 212, 220, and feminine region section 214, 222 all are fabricated from absorbent material 236. The absorbent material 236, in one embodiment, has a thickness within the range of 1/32-⅛ of an inch.

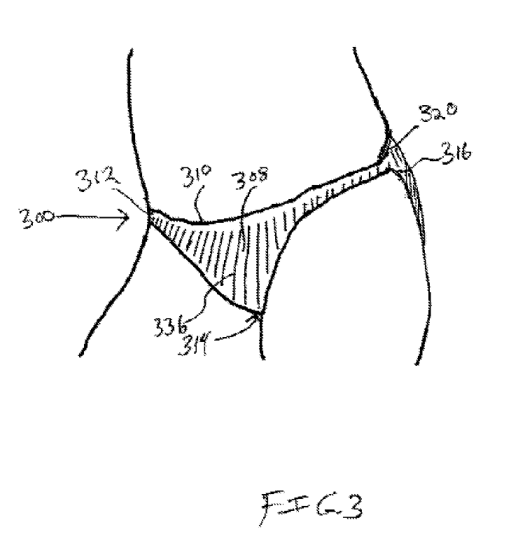

FIG. 3 shows how a disposable absorbent undergarment 300 according to one embodiment of the present invention contours a woman's body substantially similar to a convention pair of non-disposable panties. For example, FIG. 3 shows the undergarment 300 comprising the absorbent material 336 discussed above. The body regions 308, 316, waist regions 310 (and 318 not shown), hip regions 312, 320, and feminine region section 314 (and 322 not shown) are configured to contour a woman's body so that the disposable absorbent undergarment 300 resembles a conventional pair of non-disposable woman's panties. Therefore, bulges and unnatural panty lines do not occur.

The absorbent material may, for example, comprise a super absorbent polymer (SAP), a pulp or other suitable absorbent material. The super absorbent polymer (SAP), a pulp or other suitable absorbent material may be interposed between a top sheet and a back sheet. Preferably, the top sheet is preferably liquid permeable and the back sheet is liquid impermeable. One example of an absorbent material applicable to various embodiments of the present invention is an absorbent material from OUT! International, Inc.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments, and it is intended that the appended claims cover any and all such applications, modifications, and embodiments within the scope of the present invention.

What is claimed is:

1. A disposable absorbent undergarment comprising:
a front portion;
a back portion;
a crotch portion coupling the front portion and back portion together, wherein the front portion and back portion are coupled together thereby forming a waist opening and a leg opening to each side of the crotch portion;
absorbent material disposed across substantially an entire surface area of the front portion, an entire surface area of the back portion, and an entire surface area of the crotch portion;
wherein the front portion, back portion, and crotch portion with absorbent material disposed thereon comprise contours that substantially conform to an individual's body and comprise a configuration that reduces bulk under clothing.

2. The disposable absorbent undergarment of claim 1, further comprising:
a material comprising elastic properties disposed at an upper region of the back and front portions.

3. The disposable absorbent undergarment of claim 1, further comprising:
a material comprising elastic properties disposed about the leg openings.

4. The disposable absorbent undergarment of claim 1, wherein a thickness of the front portion, back portion, and crotch portion with absorbent material disposed thereon is substantially similar to conventional non-disposable women's panties.

5. The disposable absorbent undergarment of claim 1, wherein the front portion, back portion, and crotch portion consist essentially of absorbent material.
